SEQUENCE LISTING (1 sequence)

SEQ ID NO: 1 - protein, 9 aa, Artificial Sequence
  Synthesized
FGESAGAAS

(19) United States
(12) Patent Application Publication
(10) Pub. No.: US 2015/0099864 A1
(43) Pub. Date: Apr. 9, 2015

(54) PREPARATION OF ORGANOPHOSPHATE PEPTIDE ADDUCTS

(71) Applicant: BATTELLE MEMORIAL INSTITUTE, COLUMBUS, OH (US)

(72) Inventors: BOBBY N. BREWER, LEWIS CENTER, OH (US); ERIC A. LUCAS, COLUMBUS, OH (US); NATHAN T. BURKITT, COLUMBUS, OH (US)

(21) Appl. No.: 14/515,678

(22) Filed: Oct. 16, 2014

Related U.S. Application Data
(63) Continuation-in-part of application No. PCT/US14/58196, filed on Sep. 30, 2014.
(60) Provisional application No. 61/888,057, filed on Oct. 8, 2013.

(51) Int. Cl. C07K 16/44; C12N 9/18

(52) U.S. Cl. CPC C07K 16/44; C12N 9/18; C12Y 301/01008; C07K 2319/30
USPC 530/387.3; 435/68.1

(57) ABSTRACT

Disclosed are methods for the synthesis of organophosphorus-adducted peptides. Such peptides may be useful as biomarkers of organophosphate exposure, or for the synthesis of antibodies. Also disclosed are peptide adducts including peptide adducts of tabun, and aged adducts.

### CROSS REFERENCE TO RELATED APPLICATIONS

This application is a continuation in part of PCT/US14/58196 filed Sep. 30, 2014, which claims the benefit of and priority to U.S. Patent Application Ser. No. 61/888,057, filed Oct. 8, 2013, the contents of which are incorporated in its entirety by reference herein for all purposes.

### BACKGROUND

Small molecules such as organophosphates or organophosphorus nerve agents (OPNAs) are synthetic molecules used as pesticides and chemical warfare agents. Because of the risk of public exposure to such agents and the severe impact on health due to such exposure, there is a need for assays capable of rapidly identifying and/or quantifying exposure so that the extent of the impact can be determined and adequate countermeasures can be deployed. Conversely, such assays may be useful in confirming that no such exposure has occurred. While specific diagnostic assays to identify and measure exposure to organophosphates and/or OPNAs have been developed, there is a need for improved assays and methods of making such assays.

OPNAs function by reacting with an active-site serine residue of cholinesterase (Serine-198 in human butyrylchlolinesterase, for example) rendering the cholinesterase ineffective for acylcholine conversion, which is critical to nerve function. Cholinesterase can be digested with proteases such as pepsin, to generate (among other things) a well-defined nonapeptide containing the Ser-198 residue (195-FGESAGAAS) (SEQ ID NO: 1) (D. Noort, A. Fidder, M. J. van der Schans, A. G. Hulst Analytical Chemistry 2006, 78 (18), 6640-6644, DOI: 10.1021/ac060954t). This nonapeptide can be analyzed by a variety of methods, such as liquid chromatography-mass spectrometry, and thus serves as a biomarker for OPNA exposure. Analytical standards are thus required for these OPNA-adducted cholinesterase nonapeptide biomarkers.

While methods exist in the art for OPNA-adducted peptides that may be used as analytical standards, there remains a need for improved methods of production. The following disclosure seeks to address one or more needs in the art.

### BRIEF SUMMARY

Disclosed are compositions comprising organophosphorus-adducted peptides and methods of making such organophosphorus-adducted peptides, which can be used as standards for the detection of organophosphate adducts.

### BRIEF DESCRIPTION OF THE DRAWING

While the invention is claimed in the concluding portions hereof, specific embodiments provided in the accompanying detailed description may be best understood in conjunction with the accompanying figures.

FIG. 1 depicts a chemical scheme showing a process for the synthesis of a nonapeptide adducted with methylphosphonic acid. In FIG. 1, Thf is removed using Dilute H+, t-BU is removed using neat acid, and FMOC is removed using piperidene. The peptide can then be isolated and purified using standard conditions known in the art.

FIG. 2 depicts the formation of an initial OPNA-peptide adduct, followed by conversion to an aged OPNA-peptide adduct.

### DETAILED DESCRIPTION

Previous methods for the synthesis of adducted peptide compounds have involved producing an organophosphorus-adducted amino acid, and incorporating the organophosphorus-adducted amino acid into a peptide using standard peptide synthesis methods. An example of this approach can be found in WIPO Patent Application No. 2010-039163, which discloses the synthesis of organophosphorus-adducted serines such as N-Fmoc-serinyl isopropyl methylphosphonate (which can be used to produce a biomarker for sarin exposure), N-Fmoc-serinyl pinacolyl methylphosphonate (biomarker for soman exposure), N-Fmoc-serinyl cyclohexyl methylphosphonate (biomarker for cyclosarin exposure), and N-Fmoc-serinyl ethyl methylphosphonate (biomarker for VX exposure) used with standard resin-based FMOC peptide synthesis methods for synthesizing the nonapeptide H-Phe-Gly-Glu-Ser*-Ala-Gly-Ala-Ala-Ser-OH, where Ser* is the adducted Ser-198 amino acid of cholinesterase.

The resin-based FMOC peptide synthesis methods are well known in the art. The adducted peptides produced via these methods may then be used as analytical standards for identification of biomarkers for cholinesterase poisoning by organophosphorus neurotoxins. This is described in, for example, “A High-Throughput Diagnostic Method for Measuring Human Exposure to Organophosphorus Nerve Agents” (Jennifer S. Knaack, Yingtao Zhou, Carter W. Abney, Justin T. Jacob, Samantha M. Prezioso, Katelyn Hardy, Sharon W. Lemire, Jerry Thomas, and Rudolph C. Johnson Analytical Chemistry 2012, 84 (21), 9470-9477, DOI: 10.1021/ac302301w). Alternatively, the biomarkers may be used for the synthesis of antibodies using methods well known in the art.

However, certain adducted amino acids are difficult or impossible to produce in this manner. For example, N-Fmoc-serinyl ethyl N′,N′-dimethylphosphoramidate (for use in the synthesis of biomarkers for tabun exposure) cannot be made in this manner, because it is unstable. As another example, production of N-Fmoc serinyl methylphosphonic acid (to be used in the synthesis of a biomarker for old exposure to organophosphorus neurotoxins) is difficult because of the requirement that the phosphonic acid remain protected for the duration of the peptide synthesis, whereas the carboxylic acid must be de-protected before the peptide synthesis so that it can participate in the amide bond-forming reactions of the peptide synthesis. Furthermore, this method of producing a protected amino acid, then de-protecting the amino acid and incorporating it into a peptide synthesis is inefficient relative to the method of the current invention because it involves several extra synthesis reactions and purifications of intermediates.

The current disclosure relates to novel methods for the synthesis of biomarkers that overcomes one or more deficiencies in the art.

In one aspect, a method of making an adducted peptide is disclosed. The adducted peptide may serve as a biomarker that may be used as an analytical standard for identification of individuals poisoned by organophosphorus neurotoxins. In other aspects, the biomarkers may be used for production of monoclonal antibodies. Such antibodies may be used in a field test, for example, to identify individuals poisoned by organophosphorus neurotoxins.

### Peptide

The peptide useful for the method herein may be one suited for use as a biomarker or as a reagent for the manufacture of antibodies. In one aspect, the peptide may be homologous to a region of a protein (a peptide sequence) selected from cholinesterase, hemoglobin, albumin, keratin, or combinations thereof. For example, the protein may comprise a cholinesterase, for example acetylcholinesterase, butyrylcholinesterase, or a combination thereof. In one aspect, the amino acid is homologous to a region of butyrylcholinesterase. The peptide is selected such that a target amino acid—i.e., an amino acid that is a target for being adducted to phosphorus by an organophosphate—is included in the sequence.

The peptide containing a target amino acid may be used to make a biomarker for a variety of different organophosphate compounds. In some aspects, the biomarker may include a peptide adduct of a G-series nerve agent such as tabun, sarin, soman, cyclosarin, or combinations thereof. In other aspects, the biomarker may include a peptide adduct of a V-series nerve agent such as VX, VG, VE, VM, VR, or combinations thereof. In yet other aspects, the biomarker may include a peptide adduct of an organophosphorus pesticide such as malathion, parathion, paraoxon, phorate, phorate oxone, trichlorvos, glyphosate, or gluphosinate. Other biomarkers for organophosphate compounds will be readily appreciated by one of ordinary skill in the art.

The peptide of interest may further comprise aged adducts. Initial reaction of a peptide with an OPNA leads to formation of a dialkyl alkylphosphonate, or a O,O′,N,N-tetraalkylphosphoramidate as shown in FIG. 2. Following OPNA exposure, the initial adducted nonapeptide is a useful biomarker of exposure to the OPNA. However, this initial adduct slowly hydrolyzes (“ages”) in vivo to produce a monoalkyl alkylphosphonic acid, a monoalkyl phosphoric acid, a N,N-dialkylphosphoramidic, or a phosphoric acid, also known as an “aged adduct” These acids are highly resistant to further hydrolysis, and thus are much more stable and persist in the body for several days or weeks. Detection of OPNA exposure between several hours and several days after exposure thus depends on the ability to identify and quantify these aged adducts. Examples of such aged adducts include the cholinesterase serine adducts of methylphosphonic acid (biomarker for exposure to, for example, sarin, soman, cyclosarin, VX, VM, VR or mixtures thereof), ethylphosphonic acid (biomarker for exposure to, for example, VE), ethyl phosphoric acid (biomarker for exposure to tabun), N,N-dimethylphosphoramidic acid (alternative biomarker for exposure to tabun), and phosphoric acid (alternative biomarker for exposure to tabun).

Target and/or Non-Target Amino Acids

A peptide of interest may comprise one or more “target amino acids” and one or more “non-target amino acids,” wherein both the target and/or non-target amino acid is capable of being adducted to phosphorus by a phosphonylating and/or phosphorylating agent, for example, an organophosphate. That is, such target and non-target amino acids are considered “reactive,” in contrast to “non-reactive” amino acids, which cannot generally phosphonylated or phosphorylated by an organophosphate. The target amino acid is an amino acid that is the in vivo target for phosphorylation and/or phosphonylation by an organophosphate. In contrast, the non-target amino acids may be capable of reacting with a phosphonylating and/or phosphorylating group, but are not natural targets for organophosphates.

The target amino acid and/or non-target amino acid may comprise any amino acid with sidechain containing a heteroatom that is capable of forming a stable bond to the phosphorus atom of an organophosphorus compound. For example, the target amino acid may comprise a serine, a threonine, a tyrosine, a hydroxyproline, a cysteine, a glutamate, an aspartate, a lysine, or an arginine, or combinations thereof. In some aspects, the amino acid could comprise serine, threonine, tyrosine, cysteine, or hydroxyproline. In one aspect, the target amino acid could be a serine.

For example, in the case of butyrylcholinesterase, the target amino acid is the amino acid residue that is phosphonylated by an organophosphate and constitutes Serine 198. The target amino acid may comprise a serine, a threonine, a tyrosine, a hydroxyproline, a cysteine, a glutamate, an aspartate, a lysine, or an arginine, or combinations thereof, depending on the protein of interest. For example, organophosphates have been shown to bind to Tyrosine 411 in albumin (D. Noort, A. G. Hulst, A. van Zuylen, E. van Rijssel, M. J. van der Schans Archives of Toxicology 2009, 83, 1031-1036, DOI: 10.1007/s00204-009-0456-5) The peptide comprising the target amino acid may comprise any sequence surrounding the target amino acid, such sequence being readily understood by one of skill in the art and to be dictated, in part, by the ability to obtain such sequence using molecular biology methods known in the art. The peptide may range in length, from about 3 to about 100, or from about 5 to about 75, or from about 10 to about 50 amino acids, including any combination of upper and lower ranges.

In one aspect, the phosphonylating agent is selected from nerve agents, for example organophosphate, G-series nerve agents such as tabun, sarin, soman, cyclosarin, V-Series nerve agents, including VX gas; organophosphorus pesticides such as malathion, parathion, paraoxon, phorate, phorate oxone, trichlorvos, glyphosate, or gluphosinate; and combinations thereof.

### Protecting the Target Amino Acid

In one aspect, the target amino acid may be contacted with a first protecting group to form a protected amino acid. Specifically, the target amino acid may be treated with an agent that blocks the reactive moiety of the target amino acid. The target amino acid is protected such that the protecting group can be removed under mild conditions that do not cause cleavage of the peptide from the resin, or deprotection of any of the other sidechains. The first protecting group may comprise, for example, tetrahydropyranyl ether, tetrahydrofuranyl ether, triphenylmethyl ether, substituted triphenylmethyl ethers such as 4-methoxytriphenylmethyl ether, 4,4′-dimethoxytriphenylmethyl ether, or 4,4′,4″-trimethoxytriphenylmethyl ether, or combinations thereof. Other suitable protecting groups will be readily understood by one of skill in the art. Use of these and other easily removed protecting groups are described in “Greene's Protective Groups in Organic Synthesis”, 4^{th} ed., Wuts, P. G. M., Greene, T. W. 2007, J. Wiley, Hoboken, N.J.

### Protecting the Non-Target Amino Acid

The one or more non-target amino acid may likewise comprises a serine, a threonine, a hydroxyproline, an aspartate, a cysteine, a tyrosine, a glutamate, or combinations thereof. In one aspect, the one or more non-target amino acids may be protected by functionalizing the non-target amino acid with a tert-butyl ether, a tert-butyl ester, a benzyl ether, a benzyl ester, or any other protecting group that is more difficult to remove than the easily removed protecting group on the amino acid to be phosphonylated/phosphorylated, or combinations thereof. Such protecting groups will be readily understood by one of ordinary skill in the art. Suitable protecting groups are described in, for example, “Greene's Protective Groups in Organic Synthesis”, 4^{th} ed. Wuts, G. P. M.; Greene, T. W. 2007, John Wiley & Sons, Hoboken, N.J. Protection of the amine group of each amino acids in the peptide may be accomplished with flurorenylmethoxycarbonyl (FMOC), which is used in standard solid-phase peptide synthesis, such method being readily understood by one of ordinary skill in the art. The non-target serine residues may be protected, for example, as a tert-butyl ether and non-target glutamate may be protected as a tert-butyl ester.

Peptide Synthesis incorporating the Protected Target Amino Acid

The method may further comprise the step of synthesizing the peptide using the target amino acid comprising the first protecting group to form a synthesized peptide. The basic (unadducted) peptide may be synthesized using standard resin-based synthesis methods as described in “Methods in Enzymology”, volume 289, “Solid-Phase Peptide Synthesis,” Fields, G. B., ed., 1997, Academic Press, New York, N.Y., and well-known those skilled in the art. As is well known in the art, standard protecting groups may be used on all amino acids requiring protection (the non-target amino acids), and the protected target amino acid (the amino acid which will ultimately be functionalized with the organophosphorus moiety). The synthesized peptide will then contain both the target amino acid and the non-target amino acids in a protected state.

### Selective De-Protection of the Target Amino Acid

The method may further comprise the step of selectively de-protecting the target amino acid comprising the first protecting group by contacting the synthesized peptide with a dilute acid. For example, non-limiting suitable acids may be those soluble in organic solvents and may include formic acid, acetic acid, propionic acid, butyric acid, fluoroacetic acid, difluoroacetic acid, trifluoroacetic acid, chloroacetic acid, dichloroacetic acid, or trichloroacetic acid, and combinations thereof. The exact dilution of the acid depends upon the type of acid selected and/or the protecting group used, such determination being well within the skill of one of ordinary skill in the art. For example, in one aspect, the acid may be at a concentration of from about 1% to about 25%, or from about 5% to about 20%, or from about 10 to about 15%, or any combination of upper or lower limits.

The peptide may be exposed to an acid of sufficient dilution and for a sufficient period of time such that the first protecting group is removed and the target amino acid is returned to a reactive state. In some aspects, the peptide may be exposed for about one hour to about ten days, or from about two hours to about five days, or from about five hours to about one day. The amount of time that the peptide is exposed to the dilute acid may vary and such determination is also well within the skill of one of ordinary skill in the art. For example, the peptide may be exposed to the dilute acid at a temperature of from about 0° C. to about 100° C., or from about 20° C. to about 60° C. Other means of removing the first protecting group may be used, and depend on the structure of the protecting group. These may include, for example, exposure to bases such as triethylamine, diisopropylethylamine, or sodium hydroxide, or exposure to palladium catalysts such as palladium acetate. It should be emphasized that a variety of different acids, dilutions, time frames and temperatures may be used.

### Phosphonylating/Phosphorylating the Target Amino Acid

The method may further comprise the step of reacting the target amino acid by contacting the peptide with a reactive phosphorus compound. The peptide may optionally be contacted with a catalyst, a base, or a combination thereof.

Non-limiting examples of reactive phosphorus compounds that may be used include methylphosphonic dichloride, methylphosphonic dibromide, methylphosphonic diiodide, ethylphosphonic dichloride, isopropylphosphonic dichloride, tert-butylphosphonic dichloride, phenylphosphonic dichloride, isopropyl methylphosphonyl chloride, cyclohexyl methylphosphonyl chloride, pinacolyl methylphosphonyl chloride, ethyl methylphosphonyl chloride, propyl methylphosphonyl chloride, butyl methylphosphonyl chloride, methyl phosphoryl dichloride, ethyl phosphoryl dichloride, phosphoryl trichloride, ethyl N,N-dimethylphosphoramidic chloride, and combinations thereof.

Non-limiting examples of optional catalysts that may be used include pyridine, 2-picoline, 3-picoline, 4-picoline, and 4-(dimethylamino)pyridine, and combinations thereof. In one aspect, the catalyst may comprise 4-(dimethylamino)pyridine.

Non-limiting examples of the optional base that may be used include trimethylamine, triethylamine, tripropylamine, tributylamine, diisopropylethylamine, and combinations thereof. In one aspect, the optional base may include triethylamine and diisopropylethylamine.

### De-Protecting the Non-Target Amino Acids

The method may then comprise the step of de-protecting the non-target amino acids. Methods of such are well known in the art. In one aspect, the non-target amino acids may be de-protected via contact of the peptide-resin complex with concentrated acid. Suitable non-limiting examples will be readily understood by one of ordinary skill in the art. In one aspect, the acid may be trifluoroacetic acid. The concentration of the concentrated acid may be, in some aspects, from about 50% to about 100%, or from about 80% to about 100%, or from about 85 to about 90%.

Additives can be added to the concentrated acid to scavenge reactive intermediates in the deprotection and cleavage. Examples of such additives include water, phenol, anisole, thioanisole, ethanedithiol, and dithiothreitol. The contact time with the concentrated acid may vary and may be readily determined by one of ordinary skill in the art, and may include from about one minute to about two hours, or in one aspect, about one hour.

Cleavage of the Peptide from the Resin

The peptide may then be cleaved from the resin and globally deprotected using methods that are standard for FMOC-based peptide synthesis.

### EXAMPLES

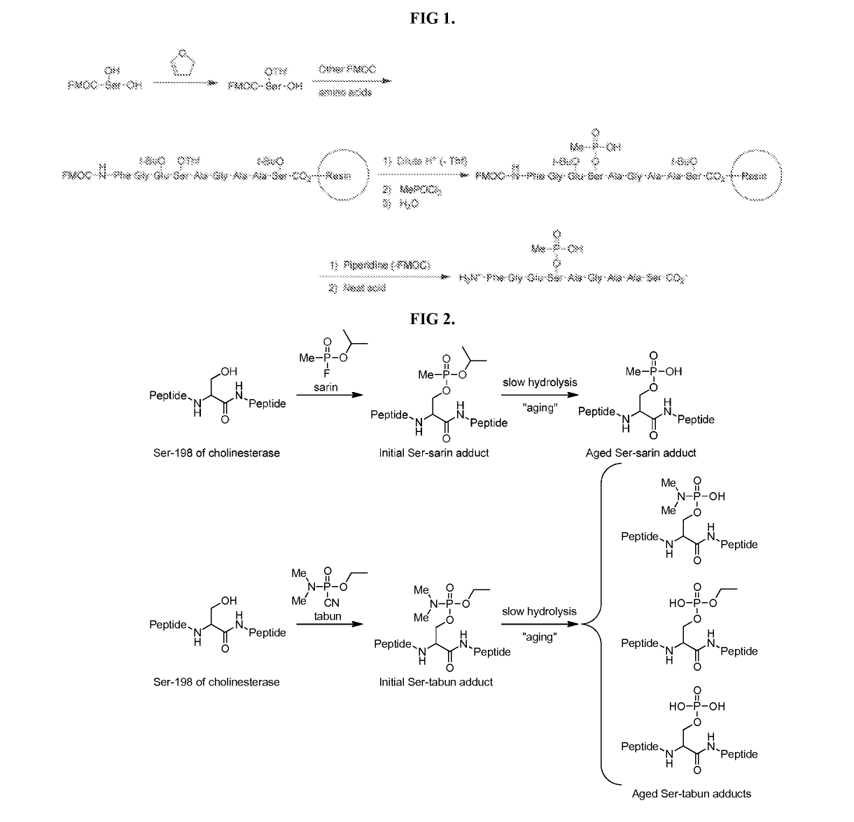

The following non-limiting examples illustrate the application of this invention to the synthesis of a phosphorus-adducted cholinesterase nonapeptide, as shown in FIG. 1.

### Example 1

### Synthesis of the Protected Serine

A slurry of FMOC-serine (3.27 g, 10.0 mmol) in dichloromethane (25 mL) was treated with dihydrofuran (0.74 g, 10.5 mmol) and pyridinium p-toluenesulfonate (0.25 g, 10 mol %), and the resulting slurry was allowed to stir at room temperature overnight. After this time, the solvent was removed under reduced pressure. The resulting viscous oil was dissolved in ethyl acetate (25 mL) and washed with water (25 mL). The ethyl acetate solution was dried over magnesium sulfate, filtered, and concentrated under reduced pressure. The resulting semi-solid oil was triturated with methyl tert-butyl ether (100 mL), and the resulting solid collected by filtration and dried under vacuum to afford 2 grams of pure N-FMOC-O-(2-tetrahydrofuranyl)serine as a white powder. A second crop was obtained by concentrating the methyl tert-butyl ether wash solution under reduced pressure and drying the residue under vacuum.

### Example 2

### Synthesis of the Protected Peptide

A peptide was prepared using a commercial, automated peptide synthesizer. The base resin was N-FMOC-O-(tert-butyl)serine-Wang resin (6.25 g, 4.0 mmol appended serine), and the amino acids used to prepare the peptide were, in order FMOC-alanine, FMOC-alanine, FMOC-glycine, FMOC-alanine, N-FMOC-O-(2-tetrahydrofuranyl)serine, FMOC-glutamic acid tert-butyl ester, FMOC-glycine, and FMOC-phenylalanine. Each coupling employed 5 equivalents of amino acid (relative to the serine loading of the resin), dissolved in DMF and activated by pretreatment with 0.8 equivalents (relative to the amino acid) of O-benzotriazol-1-yl-N,N,N′,N′-tetramethyluronium hexafluorophosphate (HBTU) in DMF and 2.0 equivalents (relative to the amino acid) of diisopropylethylamine (DIEA) in DMF/dichloromethane. Couplings were carried out for 40 minutes at room temperature. Removal of the N-terminal FMOC group before the addition of each amino acid was carried out by reaction of the peptide-resin with 20% piperidine in DMF for 20 minutes. After completion of each reaction step, the reagent solution was removed by filtration and the resin was washed thoroughly with DMF (3×). After completion of the synthesis, the peptide-resin was washed with DMF (5×100 mL), dichloromethane (5×100 mL), and methanol (5×100 mL), and the peptide-resin was dried under vacuum overnight. The yield was typically about 9 grams of peptide-resin, representing an incremental yield of approximately 2.75 grams of protected peptide (84% yield).

### Example 3

### Selective Deprotection of the Protected Peptide

The peptide-resin produced in Example 2 was slurried in tetrahydrofuran (50 mL) and treated with water (10 mL) and trifluoroacetic acid (10 mL). The resulting slurry was stirred gently and heated in a 60 C oil bath with periodic NMR analysis of the supernatant liquid diluted in DMSO-d₆ to determine the formation of 4-hydroxybutyraldehyde cyclic hemiacetal by its 1H NMR signal at δ 5.3 (dd, J1=5.0 Hz; J2=1.3). After 16 hours, the amount of 4-hydroxybutyraldehyde cyclic hemiacetal was constant, and the peptide-resin was collected by filtration, washed with tetrahydrofuran (5×50 mL) then dichloromethane (5×50 mL) then methanol (5×50 mL), and dried under vacuum overnight.

### Example 4

### Synthesis of the Phosphonylated Peptide

The selectively deprotected peptide-resin produced in Example 3 was phosphonylated as follows. A quantity of the deprotected peptide-resin (1.0 g, ca 0.4 mmol) was slurried in dichloromethane (10 mL) and treated with a solution of triethylamine (4.46 g, 44 mmol) and 4-dimethylaminopyridine (0.06 g, 0.5 mmol) in dichloromethane (10 mL), followed by a solution of methylphosphonic dichloride (1.33 g, 10 mmol) in dichloromethane (10 mL). The resulting orange-brown slurry was stirred overnight under nitrogen. After this time, the peptide-resin was collected by filtration and washed with dichloromethane (5×10 mL) and THF (5×10 mL). The phosphonylated peptide was then hydrolyzed by treatment with water (2 mL, 55 mmol) and triethylamine (2 mL, 14 mmol) in THF (20 mL) for one hour at room temperature. The resulting orange-brown resin was collected by filtration, washed with THF (5×10 mL), then treated with piperidine (20% in THF, one hour, room temperature) to remove the FMOC protecting group. The peptide-resin was then collected by filtration, washed with THF (5×10 mL) and dichloromethane (5×10 mL). The peptide was cleaved from the resin by gentle agitation for one hour with a solution of phenol (0.5 g), triisopropylsilane (0.2 g), water (0.5 g) in trifluoroacetic acid (TFA, 8.8 mL). After this time, the supernatant liquid was collected by filtration, and the resin was washed with TFA (1 mL). The combined TFA solutions were chilled to 0 C, and diluted with anhydrous diethyl ether (40 mL). The precipitated peptide was collected by centrifugation, the supernatant liquid was decanted, and the pellet was washed by re-suspending it in fresh ether (5×40 mL), with centrifugation and decantation at each wash cycle. The final pellet was dried under vacuum to yield the desired phosphonopeptide. This was purified by reverse-phase liquid chromatography on a C18 column, eluting with a water/methanol gradient containing 0.1% TFA in each gradient component. Detection was by UV absorbance, at 214 nm and 254 nm.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of making a peptide adducted with a phosphorus group, comprising the steps of
a. identifying a peptide comprising one or more target amino acids, wherein said one or more target amino acid is capable of being phosphonylated and/or phosphorylated by an organophosphate;
b. contacting said target amino acid to form a target amino acid comprising a first protecting group;
c. synthesizing said peptide using said target amino acid comprising a protecting group to form a synthesized peptide;
d. de-protecting said target amino; and
e. phosphonylating or phosphorylating the target amino acid.

2. The method of claim 1 wherein said peptide comprises one or more non-target amino acids.

3. The method of claim 2 wherein said method comprises the step of contacting said one or more non-target amino acid with a second protecting group.

4. The method of claim 3 wherein said second protecting group is selected from a tert-butyl ether, a tert-butyl ester, a benzyl ether, a benzyl ester, or combinations thereof.

5. The method of claim 3 wherein said method comprises the step of de-protecting said one or more non-target amino acids, wherein said step of de-protecting said one or more non-target amino acids occurs after said phosphonylating or phosphorylating step.

6. The method claim 1 wherein said amino acid comprises a peptide sequence homolgous to cholinesterase, hemoglobin, albumin, or combinations thereof.

7. The method of claim 5 wherein said cholinesterase comprises acetylcholinesterase, butylcholinesterase, or combinations thereof, preferably butylcholinesterase.

8. The method of claim 1 wherein said phosphonylating agent is selected from organophosphates, nerve agents (organophosphate, G-series nerve agents such as tabun, sarin, soman, cyclosarin, V-Series nerve agents, including VX gas), organophosphorus pestices (malathion, parathion, paraoxon), and combinations thereof.

9. The method of claim 1 wherein said target amino acid is Serine 198 of butylcholinesterase.

10. The method of claim 1, wherein said target amino acid comprises a serine, a threonine, an aspartate, a cysteine, a tyrosine, a glutamate, or combinations thereof.

11. The method of claim 1 wherein said first protecting group is selected from tetrahydropyranyl ether, tetrahydrofuranyl ether, triphenylmethyl ether, substituted triphenylmethyl ethers such as 4-methoxytriphenylmethyl ether, 4,4′-dimethoxytriphenylmethyl ether, or 4,4′,4″-trimethoxytriphenylmethyl ether, and combinations thereof

12. The method of claim 2, wherein said non-target amino acid comprises a serine, a threonine, an aspartate, a cysteine, a tyrosine, a glutamate, or combinations thereof.

13. The method of claim 2, wherein said non-target amino acids are protected by functionalizing said non-target amino acid with a tert-butyl ether, a tert-butyl ester, or combinations thereof.

14. The method of claim 1 wherein said synthesizing step comprises a FMOC-resin based method.

15. The method of claim 1 wherein said phosphonylating and/or phosphorylating step comprises contacting said synthesized peptide with methylphosphonyl dichloride.

16. The method of claim 1 wherein said one or more target amino acids comprise serine, and wherein said phosphonylating and/or phosphorylating comprises contacting said synthesized peptide with methylphosphonyl dichloride to produce a serinyl methylphosphonochlorate.

17. A biomarker comprising an adducted peptide manufactured according to any preceding claim.

18. An antibody comprising the adducted peptide manufactured according to claim 1.

19. The antibody of claim 18 wherein said antibody is a monoclonal antibody.
